(19) United States
(12) Patent Application Publication
(10) Pub. No.: US 2022/0125804 A1
(43) Pub. Date: Apr. 28, 2022

(54) SUPPLEMENTS CAPABLE OF ADDRESSING SIDE EFFECTS OF PRESCRIPTION DRUGS

(71) Applicant: RxSideFx, LLC, Ivins, UT (US)

(72) Inventor: Megan Milne, Ivins, UT (US)

(21) Appl. No.: 17/464,629

(22) Filed: Sep. 1, 2021

Related U.S. Application Data
(60) Provisional application No. 63/047,065, filed on Jul. 1, 2020.

(51) Int. Cl. A61K 31/573; A61K 45/06; A61K 31/155

(52) U.S. Cl. CPC A61K 31/573; A61K 31/155; A61K 45/06

(57) ABSTRACT

A nutritional supplement formulated for use in conjunction with a particular prescription drug includes a combination of ingredients that addresses a combination of side effects of the prescription drug. The ingredients of the nutritional supplement may address all treatable very common side effects and/or common side effects of the prescription drug or the ingredients may address treatable serious side effects of the prescription drug. Nutritional supplement-prescription drug combinations that reduce or prevent the occurrence of side effects are also disclosed, as are methods for treating the side effects of prescription drugs with nutritional supplements.

### CROSS-REFERENCE TO RELATED APPLICATION

A claim for priority to the Jul. 1, 2020 filing date of U.S. Provisional Patent Application No. 63/047,065, titled SUPPLEMENTS CAPABLE OF ADDRESSING SIDE EFFECTS OF PRESCRIPTION DRUGS (“the '065 Provisional application”), is hereby made pursuant to 35 U.S.C. § 119(e). The entire disclosure of the '065 Provisional application is hereby incorporated herein.

### TECHNICAL FIELD

This disclosure relates generally to supplements and, more specifically, to supplements that are tailored for use with a specific drug and formulated to address (e.g., counteract, treat, etc.) one or more side effects of the drug (e.g., a prescription drug, etc.). A supplement according to this disclosure may be administered to an individual in conjunction with a drug.

### RELATED ART

Approximately 0.5-1% of the United States population is taking prednisone long-term, many of whom are experiencing side effects. According to ClinCalc online, 22.5 million prescriptions for prednisone were dispensed in the U.S. in 2015, making it the 32nd most frequently prescribed medication in the U.S. in 2015. Unlike newly-approved medications for which the U.S. Federal Drug Administration (FDA) requires a battery of tests to show which side effects are most common, prednisone is an old drug, approved in 1955, and no manufacturer was ever required to test for side effects. The FDA-certified drug package insert contains only a small selection of side effects and does not include some of the most commonly experienced issues, such as “moon face.” Therefore, while prednisone is known to have many bothersome side effects, the only data available about prednisone's side effects is in independent research.

Another common medication for which there are many bothersome side effects is metformin, which is used to treat diabetes. Approximately 9 million Americans have diabetes; metformin is the first-line therapy to treat diabetes. According to ClinCalc online, 84 million prescriptions were filled for metformin in the U.S. in 2015, making it the 4th most prescribed drug in the U.S. that year. Although metformin is an effective treatment for diabetes, it is responsible for many adverse side effects in patients who take it.

The beta-blocker drug class is also known to have bothersome side effects. Beta-blockers are used to treat high blood pressure and heart failure, and to aid in recovery from heart attacks. Approximately one third of Americans use beta-blockers to treat high blood pressure. According to ClinCalc online, 70 million prescriptions were dispensed in the U.S. in 2015 for metoprolol, the most commonly used beta-blocker, making it the 8th most prescribed drug in the U.S. that year. Other beta-blockers that are commonly used in the U.S. include carvedilol, with 25 million prescriptions, the 26th most common drug in 2015; atenolol with 30 million prescriptions, the 17th most common drug in 2015; and 13.8 million prescriptions for propranolol, making it the 63rd most prescribed drug in 2015. Approximately ten percent of patients who take beta-blockers experience adverse side effects from.

Atypical antipsychotic drugs, which often work well in the treatment of mental illnesses, such as schizophrenia, bipolar disorder, and depression, also have many adverse side effects. Approximately 1 in 25 Americans have a serious mental illness. According to ClinCalc online, 9 million prescriptions for quetiapine were dispensed in the U.S. in 2015, making it the 87th most prescribed drug in the U.S. in 2015. The other antipsychotic drugs most commonly used in the U.S. in 2015 include risperidone with 5.6 million prescriptions, the 130th most common drug; aripiprazole with 5.4 million prescriptions, the 133rd most common drug; and 2.4 million prescriptions for olanzapine dispensed in 2015, making it the 227th most prescribed drug. Approximately ten percent of these patients also experience adverse side effects from their prescriptions.

The anti-seizure drug class, also known as anticonvulsants or antiepileptic drugs, also cause many adverse side effects in patients to whom these drugs are administered. Approximately 1% of the American population has epilepsy and is receiving anti-seizure therapy long-term. According to ClinCalc online, there were 4.1 million prescriptions were dispensed for phenytoin (Dilantin) in the U.S. in 2015, making it the 158th most prescribed drug in the U.S. in 2015. Another similar drug, divalproex sodium (Depakote), was dispensed 6.1 million times in the U.S. in 2015, making it the 120th most often prescribed drug in the U.S. in 2015. Carbamazepine was dispensed nearly 2 million times in the U.S. in 2015, making it the 260th most frequently prescribed drug. Phenobarbital is another, older anti-seizure drug that is sometimes administered to epilepsy patients.

Almost all prescription drugs cause some adverse side effects in at least some of the patients to whom the prescription drugs are administered. Thus, there is a great need for safe and effective ways to address the side effects of prescription drugs.

### SUMMARY

A supplement according to this disclosure may be tailored to safely and effectively address one or more side effects of one or more drugs. As used herein, the phrase “side effects” refers to adverse side effects that are known to be caused by a particular drug. The term “drug,” as used herein, includes prescription drugs.

In some embodiments, a supplement of this disclosure may be tailored for use in conjunction with a particular type of drug, or even for use in conjunction with a particular drug. Some non-limiting examples of drugs for which a supplement according to this disclosure may be tailored include prednisone, anti-seizure drugs (e.g., phenytoin, divalproex sodium, carbamazepine, phenobarbital, etc.), metformin, beta-blockers (e.g., metoprolol, carvedilol, atenolol, propranolol, etc.), and atypical antipsychotic drugs (e.g., quetiapine, risperidone, aripiprazole, olanzapine, etc.).

The supplement may comprise a so-called dietary supplement or nutritional supplement, and may include one or more vitamins, minerals, natural products, or combinations thereof. The supplement may have a formulation (e.g., one or more ingredients in amounts, etc.) that may address, or treat, one or more side effects of the drug. The ingredients of the supplement may be selected to address one or more of the most common side effects of the drug. In some embodiments, a supplement may include a combination of ingredients that address all of the very common side effects of a drug (i.e., side effects occurring in at least 10% of individuals who receive the drug) and/or all of the treatable common side effects of the drug (i.e., side effects occurring in at least 1% of individuals who receive the drug). A supplement may include one or more ingredients that have been selected to address one or more or all of the treatable serious side effects of the drug (i.e., side effects that result in birth defects, require hospitalization, cause permanent damage or disability, are life-threatening, or result in death). In some embodiments, the ingredients of the supplement may be selected to address each treatable known side effect of the drug.

As another option, a supplement may include, as ingredients, all of the nutrients (e.g., vitamins, minerals, etc.) that are known to be depleted by a drug that corresponds to the supplement. In another option, a supplement may include ingredients that are most effective for treating side effects of the corresponding drug. As yet another option, a supplement may include a combination of nutrients that are known to be depleted by a corresponding drug and ingredients that are most effective for treating side effects of the corresponding drug.

The amount of each ingredient in the supplement may correspond to a dose of that ingredient that will address a particular side effect of a drug or to a recommended daily allowance (RDA) of that ingredient. Of course, the number of units (e.g., pills, capsules, gel-caps, etc.) per dose and the number of doses per day may also be taken into consideration in determining the amount of each ingredient in each unit of a supplement according to this disclosure.

A supplement according to this disclosure may be formulated not to interfere with a beneficial activity of the prescription drug and/or beneficial effect of the prescription drug on an individual's body. Without limitation, the effects of the ingredients, the amounts of the ingredients, and combinations of ingredients may be considered in formulating a supplement that will address the side effects of a particular drug without adversely affecting the beneficial effects of the drug.

The ingredients of the supplement may comply with United States Pharmacopeia (USP) standards.

A drug-supplement combination includes a prescription drug capable of treating a medical condition and a supplement with a combination of ingredients capable of addressing a plurality of side effects of the prescription drug. The supplement may have any of the above-identified characteristics or features of any combination of the above-identified characteristics or features. Such a drug-supplement combination may be useful for reducing or preventing the side effects associated with administration of a prescription drug.

In a method according to this disclosure, a drug capable of treating a medical condition may be administered to an individual who suffers from the medical condition. As used herein, the term “administer” includes self-administration of the drug, or taking the drug, and administration of the drug by another individual, such as healthcare professional (e.g., a physician or another qualified healthcare practitioner) or other caretaker.

The drug may be administered to the individual in accordance with a prescription by a healthcare provider. A supplement may be administered to the individual in conjunction with administration of the drug. The supplement may be tailored to address one or more known side effects of the drug (e.g., one or more common side effects of the drug, one or more of the most severe side effects of the drug, a combination of common and severe side effects of the drug, all known side effects of the drug, etc.). The supplement may also be tailored to avoid any deleterious effects on the beneficial activity of the drug and/or the potential benefits of the drug to the individual to whom the drug is administered.

A supplement may be administered to an individual once a day (e.g., once each day a corresponding drug is administered, etc.) or a plurality of times each day (e.g., each time the corresponding drug is administered, regardless of the number of times the drug is prescribed to be administered or is actually administered, etc.). The supplement may be administered to an individual concurrently with (e.g., simultaneously, at about the same time of day as, etc.) administration of the corresponding drug to the individual. Alternatively, the supplement may be administered to an individual at a different time of day than administration of the corresponding drug to the individual. For example, the supplement may be administered to the individual within a predetermined period of time before side effects of the corresponding drug are expected to occur, at a time of day when side effects of the corresponding drug are expected to occur, or shortly after side effects of the corresponding drug occur. As an example, a supplement may be administered at about the same time of day the corresponding drug is administered to address one or more side effects that occur while the effects of the corresponding drug are strongest. A supplement may be administered when the effects of the corresponding drug start to wear off or when the effectiveness of the drug is expected to end to address one or more side effects that occur as the effects of a corresponding drug diminish or end. In some embodiments, different supplements may be administered to address different side effects of a corresponding drug that appear or that are most prevalent at different times. Administration of the supplement may follow a predetermined dosing schedule.

Other aspects of the disclosed subject matter, as well as features and advantages of the disclosed subject matter, will become apparent to those of ordinary skill in the art through consideration of the foregoing disclosure, the ensuing description, and the appended claims.

### DETAILED DESCRIPTION

Various embodiments of supplements that are tailored for use with certain types of drugs or with specific drugs and that are formulated to address the side effects of those drugs are disclosed.

Prednisone.

TABLE 1
Effective and likely effective therapies for prednisone side effects.

| Therapy | Side Effect | Effectiveness |
|---|---|---|
| Calcium | Osteoporosis | Likely Effective |
| Magnesium | Dyspepsia | Effective |
| Melatonin | Insomnia | Likely Effective |
| Niacin | Dyslipidemia | Likely Effective |
| Vitamin B6 | Anemia | Effective |
| Vitamin D | Corticosteroid-induced | Likely Effective |
| | osteoporosis | |
| Zinc | Diarrhea | Likely Effective |

Expanding the list to include therapies that may be effective (i.e., “Possibly Effective” therapies) covered all of the top prednisone side effects with at least one natural remedy, except for “moon face” for which there are no remedies aside from discontinuing prednisone consumption.

TABLE 2
Possibly effective therapies for prednisone side effects.

| Therapy | Side Effect | Effectiveness |
|---|---|---|
| Berberine | Diabetes | Possibly Effective |
| Berberine | Hyperlipidemia | Possibly Effective |
| Berberine | Osteoporosis | Insufficient Reliable |
| | | Evidence |
| Berberine | Thrombocytopenia | Insufficient Reliable |
| | | Evidence |
| Calcium | Dyspepsia | Effective |
| Calcium | Hypertension | Possibly Effective |
| Calcium | Weight gain | Possibly Effective |
| Calcium | Osteoporosis | Likely Effective |
| Cassia | Diabetes | Possibly Effective |
| Cinnamon | | |
| Chromium | Diabetes | Possibly Effective |
| Chromium | Hyperlipidemia | Possibly Effective |
| Folic Acid | Depression | Possibly Effective |
| Folic Acid | Hypertension | Possibly Effective |
| Magnesium | Dyspepsia | Effective |
| Magnesium | Coronary artery disease | Possibly Effective |
| Magnesium | Diabetes | Possibly Effective |
| Magnesium | Hypercholesterolemia | Possibly Effective |
| Magnesium | Osteoporosis | Possibly Effective |
| Melatonin | Anxiety | Possibly Effective |
| Melatonin | Hypertension | Possibly Effective |
| Melatonin | Sleep-wake cycle disturbances | Likely Effective |
| Melatonin | Insomnia | Possibly Effective |
| Melatonin | Thrombocytopenia | Possibly Effective |
| Niacin | Dyslipidemia | Likely Effective |
| Potassium | Hypertension | Possibly Effective |
| Riboflavin | Cataracts | Possibly Effective |
| Thiamine | Cataracts | Possibly Effective |
| Vitamin A | Cataracts | Possibly Effective |
| Vitamin B6 | Anemia | Effective |
| Vitamin B6 | Seizures | Effective |
| Vitamin C | Anemia | Possibly Effective |
| Vitamin C | Erythema | Possibly Effective |
| Vitamin C | Hypercholesterolemia | Possibly Effective |
| Vitamin C | Osteoporosis | Insufficient Reliable |
| | | Evidence |
| Vitamin D | Heart failure | Possibly Effective |
| Vitamin D | Corticosteroid-induced | Likely Effective |
| | osteoporosis | |
| Vitamin D | Osteoporosis | Likely Effective |
| Zinc | Acne | Possibly Effective |
| Zinc | Depression | Possibly Effective |
| Zinc | Diarrhea | Likely Effective |
| Zinc | Peptic ulcers | Possibly Effective |
| Zinc | Osteoporosis | Possibly Effective |

Because there is diurnal (daytime, nighttime) variation in prednisone's side effects, with some side effects being more likely to occur early in the day right after dosing and other side effects being more likely later at night when the benefits of prednisone should be wearing off, a supplement that addresses one or more side effects of prednisone may be dosed twice daily. The ingredients and/or doses of each ingredient may vary from one time of day to another time of day. Thus, supplements with different formulas may be administered at different times of day. The dose of each ingredient may correspond to the Institute of Medicine's recommended daily allowance (RDA) for that ingredient and/or to a dose that is determined to be most effective for addressing at least one side effect of prednisone.

TABLE 3
Doses of ingredients in prednisone side effect dietary supplement.

| Therapy | Chemical | Dose AM | | Dose PM | |
|---|---|---|---|---|---|
| Berberine | Berberine bisulfate | 5 | mg | 5 | mg |
| Calcium | Calcium citrate | 500 | mg | 500 | mg |
| Cassia Cinnamon | Cinnamomum | 120 | mg | 100 | mg |
| | aromaticum | | | | |
| Chromium | Chromium picolinate | 200 | mcg | 200 | mcg |
| Folic Acid (B9) | Pteroylglutamic acid | 200 | mcg | 200 | mcg |
| Magnesium | Magnesium oxide | 80 | mg | 80 | mg |
| Melatonin | N-Acetyl-5- | | | 2 | mg |
| | Methoxytryptamine | | | | |
| Niacin (B3) | Niacinamide ascorbate | | | 14 | mg |
| Potassium | Potassium chloride | 40 | mg | 40 | mg |
| Riboflavin (B2) | Riboflavin | 0.6 | mg | 0.6 | mg |
| Thiamine (B1) | Thiamin mononitrate | 0.8 | mg | 0.8 | mg |
| Vitamin A | Vitamin A acetate | 1500 | IU | 1500 | IU |
| Vitamin B6 | Pyridoxine hydrochloride | 1 | mg | 1 | mg |
| Vitamin C | Ascorbic acid | 40 | mg | 40 | mg |
| Vitamin D | Cholecalciferol | 400 | IU | 400 | IU |
| Zinc | Zinc oxide | 5 | mg | 5 | mg |

Dose AM outlines that strength in mg, mcg, or IU will be included in the morning dose of this supplement, taken at the same time as the daily dose of prescription prednisone.
Dose PM is the corresponding dose taken at bedtime.
The B numbers in parentheses ((B#)) correspond to the B-vitamins to which the names that precede the B numbers commonly refer.

TABLE 4
Doses of ingredients in an alternate
prednisone side effect dietary supplement.

| Therapy | Chemical | Dose AM | | Dose PM | |
|---|---|---|---|---|---|
| Berberine | Berberine bisulfate | 15 | mg | | |
| Calcium | Calcium carbonate | 500 | mg | 500 | mg |
| Cassia Bark | Cinnamomum | 60 | mg | 60 | mg |
| | aromaticum | | | | |
| Chromium | Chromium picolinate | 200 | mcg | 200 | mcg |
| Folic Acid (B9) | Pteroylglutamic acid | 200 | mcg | 200 | mcg |
| Magnesium | Magnesium oxide | 100 | mg | 80 | mg |
| Melatonin | N-Acetyl-5- | | | 2 | mg |
| | Methoxytryptamine | | | | |
| Niacin (B3) | Niacinamide ascorbate | | | 16 | mg |
| Potassium | Potassium chloride | 40 | mg | 40 | mg |
| Riboflavin (B2) | Riboflavin | 1.3 | mg | | |
| Thiamine (B1) | Thiamin mononitrate | 1.5 | mg | | |
| Vitamin A | Vitamin A acetate | 452 | mcg | 452 | mcg |
| Vitamin B6 | Pyridoxine | | | 1.7 | mg |
| | hydrochloride | | | | |
| Vitamin C | Ascorbic acid | 45 | mg | 45 | mg |
| Vitamin D | Cholecalciferol | 10 | mcg | 10 | mcg |
| Vitamin K | Menaquinone-7 | | | | |
| Zinc | Zinc oxide | 11 | mg | | |

Dose AM outlines that strength in mg, mcg, or IU will be included in the morning dose of this supplement, taken at the same time as the daily dose of prescription prednisone.
Dose PM is the corresponding dose taken at bedtime.
The B numbers in parentheses ((B#)) correspond to the B-vitamins to which the names that precede the B numbers commonly refer.

TABLE 5
Doses of ingredients in another alternate
prednisone side effect dietary supplement.

| Therapy | Chemical | Dose AM | | Dose PM | |
|---|---|---|---|---|---|
| Calcium | Calcium citrate | 500 | mg | 500 | mg |
| Chromium | Chromium picolinate | 200 | mcg | 200 | mcg |
| Folic Acid (B9) | Pteroylglutamic acid | 200 | mcg | 200 | mcg |
| Magnesium | Magnesium oxide | 100 | mg | 100 | mg |
| Niacin (B3) | Niacinamide ascorbate | | | 16 | mg |
| Potassium | Potassium chloride | 40 | mg | 40 | mg |
| Proprietary Blend | Cinnamomum | 75 | mg | | |
| of Cassia Bark | aromaticum | | | | |
| and Berberine | Berberine bisulfate | | | | |
| (from Indian | | | | | |
| Barberry root) | | | | | |
| Proprietary Blend | Cinnamomum | | | 62 | mg |
| of Cassia Bark | aromaticum | | | | |
| and Melatonin | N-Acetyl-5- | | | | |
| | Methoxytryptamine | | | | |
| Riboflavin (B2) | Riboflavin | 1.3 | mg | | |
| Thiamine (B1) | Thiamin mononitrate | 1.5 | mg | | |
| Vitamin A | Vitamin A acetate | 452 | mcg | 452 | mcg |
| Vitamin B6 | Pyridoxine | | | 1.7 | mg |
| | hydrochloride | | | | |
| Vitamin C | Ascorbic acid | 45 | mg | 45 | mg |
| Vitamin D | Cholecalciferol | 10 | mcg | 10 | mcg |
| Zinc | Zinc oxide | 11 | mg | | |

| | | |
|---|---|---|
| Microcrystalline | Present | Present |
| cellulose | | |
| Magnesium | Present | Present |
| Stearate | | |
| Silicon dioxide | Present | Present |

Dose AM outlines that strength in mg, mcg, or IU will be included in the morning dose of this supplement, taken at the same time as the daily dose of prescription prednisone.
Dose PM is the corresponding dose taken at bedtime.
The B numbers in parentheses ((B#)) correspond to the B-vitamins to which the names that precede the B numbers commonly refer.

Metformin

TABLE 6

| Therapy | Side Effect |
|---|---|
| Berberine | Diabetes |
| Biotin (B7) | Weight gain, hyperglycemia |
| Calcium | B12 deficiency, dyspepsia, |
| | osteoporosis, hypertension, |
| | weight gain |
| Cassia Cinnamon | Diabetes, hypertension |
| Chromium | Diabetes, anorexia |
| Coenzyme Q10 | Headache |
| Folic Acid (B9) | Depression |
| Ginger | Vomiting, indigestion |
| Magnesium | Constipation, dyspepsia, |
| | insulin resistance |
| Riboflavin (B2) | Headache |
| Thiamine (B1) | Diabetic nephropathy |
| Vitamin B6 | Nausea |
| Vitamin B12 | B12 deficiency, peripheral |
| | neuropathy |
| Vitamin D | Osteoporosis, diabetes |
| Zinc | Diarrhea, indigestion, |
| | metallic taste, infection |

Folic Acid (B9), Thiamine (B1), and Vitamin B12 therapies are nutrients depleted by metformin.
Magnesium and Zinc therapies are believed to be most effective for addressing one or more side effects of metformin.
The B numbers in parentheses ((B#)) correspond to the B-vitamins to which the names that precede the B numbers commonly refer.

This supplement may be dosed once a day to simplify a likely already-complicated medication regimen for diabetes. The dose of the ingredients may correspond to the Institute of Medicine's recommended daily allowance (RDA) for each ingredient and/or to a dose of each ingredient that is determined to be most effective at addressing at least one side effect of metformin.

TABLE 7
Dose of ingredients in metformin side effect dietary supplement.

| Therapy | Chemical | Dose | |
|---|---|---|---|
| Berberine | Berberine bisulfate | 5 | mg |
| Biotin (B7) | Cis-hexahydro-2-oxo-1H- | 1 | mg |
| | thieno[3,4-d]-imidazole-4-valeric | | |
| | acid | | |
| Calcium | Calcium citrate | 500 | mg |
| Cassia Cinnamon | Cinnamomum aromaticum | 100 | mg |
| Chromium | Chromium picolinate | 200 | mcg |
| Coenzyme Q10 | Ubiquinone | 30 | mg |
| Folic Acid (B9) | Pteroylglutamic acid | 400 | mcg |
| Ginger | Zingiber officinale | 100 | mg |
| Magnesium | Magnesium oxide | 80 | mg |
| Riboflavin (B2) | Riboflavin | 1.3 | mg |
| Thiamine (B1) | Thiamin mononitrate | 1.5 | mg |
| Vitamin B6 | Pyridoxine hydrochloride | 2 | mg |
| Vitamin B12 | Cyanocobalamin | 50 | mcg |
| Vitamin D | Cholecalciferol | 200 | IU |
| Zinc | Zinc oxide | 10 | mg |

Dose outlines the strength in mg, mcg, or IU that will be included in the daily dose of this supplement.
The B numbers in parentheses ((B#)) correspond to the B-vitamins to which the names that precede the B numbers commonly refer.

Beta-Blockers

TABLE 8
Natural therapies for beta-blocker side effects and nutrient depletions.

| Therapy | Side Effect |
|---|---|
| Biotin (B7) | Weight gain, hyperglycemia |
| Calcium | Hypertension, weight loss |
| Cassia Cinnamon | Diabetes, hypertension |
| Chromium | Weight gain, hyperglycemia |
| Coenzyme Q10 | Headache, hypertension |
| Folic Acid (B9) | Hypertension, depression |
| Ginger | Vertigo, nausea, diabetes, hyperglycemia |
| Magnesium | Constipation, nausea, insulin resistance |
| Melatonin | Insomnia, hypertension, confusion |
| Riboflavin | Headache |
| Thiamine (B1) | Glucose intolerance |
| Vitamin B6 | Nausea |
| Vitamin B12 | Drowsiness |
| Vitamin C | Hypertension |
| Zinc | Diarrhea, depression, pruritus |

Melatonin is depleted by beta-blockers. Magnesium and Vitamin B6 are believed to be most effective for addressing one or more side effects.
The B numbers in parentheses ((B#)) correspond to the B-vitamins to which the names that precede the B numbers commonly refer.

This supplement may be dosed once a day at bedtime to simplify a likely already-complicated medication regimen for high blood pressure, heart disease, or a patient who has suffered a heart attack. The dose of each ingredient may correspond to the Institute of Medicine's recommended daily allowance (RDA) for that ingredient and/or to a dose that is determined to be most effective for addressing at least one side effect of a beta-blocker

TABLE 9
Dose of ingredients in beta-blocker side effect dietary supplement.

| Therapy | Chemical | Dose | |
|---|---|---|---|
| Biotin (B7) | Cis-hexahydro-2-oxo-1H- | 2 | mg |
| | thieno[3,4-d]-imidazole-4-valeric | | |
| | acid | | |
| Calcium | Calcium citrate | 250 | mg |
| Cassia Cinnamon | Cinnamomum aromaticum | 100 | mg |
| Chromium | Chromium picolinate | 600 | mcg |
| Coenzyme Q10 | Ubiquinone | 30 | mg |
| Folic Acid (B9) | Pteroylglutamic acid | 1 | mg |
| Ginger | Zingiber officinale | 100 | mg |
| Magnesium | Magnesium oxide | 80 | mg |
| Melatonin | N-Acetyl-5-Methoxytryptamine | 2.5 | mg |
| Riboflavin | Riboflavin | 1.3 | mg |
| Thiamine (B1) | Thiamin mononitrate | 1.5 | mg |
| Vitamin B6 | Pyridoxine hydrochloride | 2 | mg |
| Vitamin B12 | Cyanocobalamin | 50 | mcg |
| Vitamin C | Ascorbic acid | 30 | mg |
| Zinc | Zinc oxide | 10 | mg |

Dose outlines the strength in mg, mcg, or IU that will be included in the daily dose of this supplement.
The B numbers in parentheses ((B#)) correspond to the B-vitamins to which the names that precede the B numbers commonly refer.

Antipsychotic Drugs

TABLE 10
Natural therapies for antipsychotic side effects.

| Therapy | Side Effect |
|---|---|
| American Ginseng | Schizophrenia, working memory, abnormal |
| | gait, muscle rigidity, hypertension, diabetes |
| Biotin (B7) | weight gain, hyperglycemia |
| Calcium | Hypertension, weight gain |
| Cassia Cinnamon | Diabetes, hypertension |
| Folic Acid (B9) | Hypertension, depression, schizophrenia, |
| | diabetes, mania |
| Ginger | Vertigo, dyspepsia, nausea, vomiting, |
| | diabetes, hyperglycemia |
| Lemon Balm | Dyspepsia, insomnia, anxiety, insomnia |
| L-theanine | Cognitive performance, anxiety |
| Magnesium | Constipation, abdominal pain, agitation, |
| | drowsiness, fatigue, insulin |
| | resistance, dyspepsia |
| Passion Flower | Anxiety, insomnia |
| Thiamine (B1) | Glucose intolerance |
| Vitamin B12 | Fatigue, atherosclerosis, diarrhea, |
| | hypertriglyceridemia, schizophrenia |
| Vitamin B6 | Tardive dyskinesia, atherosclerosis, |
| | nausea, vomiting, tremor |
| Vitamin C | Hypertension, oxidative stress in schizophrenia |
| Zinc | Diarrhea, depression, urinary incontinence |

The B numbers in parentheses ((B#)) correspond to the B-vitamins to which the names that precede the B numbers commonly refer.

This supplement may be dosed once a day at bedtime to simplify a likely already-complicated medication regimen for treating a mental illness. The dose of each ingredient may correspond to the Institute of Medicine's recommended daily allowance (RDA) for that ingredient and/or to a dose of that ingredient that is determined to be most effective for addressing one or more side effects of an antipsychotic drug.

TABLE 11
Dose of ingredients in antipsychotic side effect dietary supplement.

| Therapy | Chemical | Dose | |
|---|---|---|---|
| American Ginseng | Panax quinquefolius. | 100 | mg |
| Biotin (B7) | Cis-hexahydro-2-oxo-1H- | 2 | mg |
| | thieno[3,4-d]-imidazole-4-valeric | | |
| | acid | | |
| Calcium | Calcium citrate | 100 | mg |
| Cassia Cinnamon | Cinnamomum aromaticum | 100 | mg |
| Folic Acid (B9) | Pteroylglutamic acid | 1 | mg |
| Ginger | Zingiber officinale | 100 | mg |
| Lemon Balm | Melissa officinalis | 60 | mg |
| L-theanine | 5-N-ethylglutamine | 12 | mg |
| Magnesium | Magnesium oxide | 80 | mg |
| Passion Flower | Passiflora incarnata | 40 | mg |
| Thiamine (B1) | Thiamin mononitrate | 1.5 | mg |
| Vitamin B12 | Cyanocobalamin | 25 | mcg |
| Vitamin B6 | Pyridoxine hydrochloride | 2 | mg |
| Vitamin C | Ascorbic acid | 75 | mg |
| Zinc | Zinc oxide | 10 | mg |

Dose outlines the strength in mg, mcg, or IU that will be included in the daily dose of this supplement.
The B numbers in parentheses ((B#)) correspond to the B-vitamins to which the names that precede the B numbers commonly refer.

Anti-Seizure Drugs

TABLE 12

| Therapy | Side Effect |
|---|---|
| Biotin (B7) | Weight gain, hyperglycemia, nutrient deficiency |
| Calcium | Dyspepsia, osteoporosis, hypertension, |
| | weight gain, seizures |
| Coenzyme Q10 | Migraine headache, hypertension, cerebellar |
| | ataxia, dry mouth |
| Folic Acid (B9) | Hypertension, seizures, gingival enlargement |
| Magnesium | Constipation, dyspepsia, osteoporosis, low |
| | back pain, insulin resistance |
| Melatonin | Insomnia, hypertension, tinnitus, anxiety, |
| | thrombocytopenia, seizures |
| Passion Flower | Anxiety, insomnia |
| Thiamine (B1) | Ataxia |
| Vitamin B12 | Nystagmus, drowsiness, peripheral neuropathy |
| Vitamin B6 | Pyridoxine-dependent seizures, nausea, |
| | vomiting, anemia |
| Vitamin D | Osteoporosis, osteomalacia |
| Vitamin E | Anemia, osteoporosis |
| Zinc | Diarrhea, peptic ulcers, osteoporosis, |
| | loss of taste, seizures, infections |
| German | Anxiety, diarrhea, dyspepsia |
| Chamomile | |
| Ginger | Vertigo, dyspepsia |
| Lavender | Anxiety |
| Lemon Balm | Dyspepsia, insomnia |
| Riboflavin (B2) | Migraine headache |
| Vitamin C | Hypertension, osteoporosis, infection |

Biotin, Calcium, Folic Acid (B9), Thiamine (B1), Vitamin B12, Vitamin D, Vitamin E, and Zinc are depleted by anti-seizure drugs.
Calcium, Magnesium, and Vitamin B6 are believed to be most effective for addressing one or more side effects of anti-seizure drugs.
The B numbers in parentheses ((B#)) correspond to the B-vitamins to which the names that precede the B numbers commonly refer.

Because there is diurnal variation in the side effects of many anti-seizure drugs, with some side effects more likely early in the day right after dosing, and other side effects more likely to occur later at night, this product may be dosed twice daily. The ingredients and/or doses of each ingredient may vary from one time of day to another time of day. Thus, supplements with different formulas may be administered at different times of day. The dose of each ingredient may correspond to the Institute of Medicine's recommended daily allowance (RDA) for that ingredient or to a dose that is determined to be the most effective for one or more side effects of one or more anti-seizure drugs.

TABLE 13
Dose of ingredients in antiseizure drug side effect dietary supplement.

| Therapy | Chemical | Dose AM | | Dose PM | |
|---|---|---|---|---|---|
| Biotin (B7) | Cis-hexahydro-2- | 1 | mg | 1 | mg |
| | oxo-1H-thieno[3,4-d]- | | | | |
| | imidazole- | | | | |
| | 4-valeric acid | | | | |
| Calcium | Calcium citrate | 500 | mg | 500 | mg |
| Coenzyme Q10 | Ubiquinone | 30 | mg | 30 | mg |
| Folic Acid (B9) | Pteroylglutamic acid | 200 | mcg | 200 | mcg |
| Magnesium | Magnesium oxide | 80 | mg | 80 | mg |
| Melatonin | N-Acetyl-5- | | | 2 | mg |
| | Methoxytryptamine | | | | |
| Passion Flower | Passiflora incarnata | | | 40 | mg |
| Thiamine (B1) | Thiamin mononitrate | 1.5 | mg | | |
| Vitamin B12 | Cyanocobalamin | 25 | mcg | 25 | mcg |
| Vitamin B6 | Pyridoxine | | | 2 | mg |
| | hydrochloride | | | | |
| Vitamin D | Cholecalciferol | 400 | IU | 400 | IU |
| Vitamin E | Alpha-tocopherol | 5 | mg | 10 | mg |
| Zinc | Zinc oxide | 10 | mg | | |

Dose AM outlines the strength in mg, mcg, or IU that will be included in the morning dose of this supplement, taken at the same time as the morning dose of prescription antiseizure drug.
Dose PM is the corresponding dose taken at bedtime.
The B numbers in parentheses ((B#)) correspond to the B-vitamins to which the names that precede the B numbers commonly refer.

Supplements that are tailored to address the side effects of other drugs are also within the scope of this disclosure, as are methods of administering supplements to address the side effects of other drugs.

Although the foregoing description sets forth many specifics, these should not be construed as limiting the scope of any of the claims, but merely as providing illustrations of some embodiments and variations of elements or features of the disclosed subject matter. Other embodiments of the disclosed subject matter may be devised which do not depart from the spirit or scope of any of the claims. Features from different embodiments may be employed in combination. Accordingly, the scope of each claim is limited only by its plain language and the legal equivalents thereto.

What is claimed:

1. A method for treating a medical condition, comprising:
administering a drug to an individual suffering from the medical condition in accordance with a prescription by a healthcare provider;
administering a supplement including a combination of ingredients to the individual in connection with administering the drug, the supplement tailored to address a combination of side effects of the drug.

2. The method of claim 1, wherein administering the supplement comprises administering a supplement tailored to address all treatable common side effects and treatable very common side effects of the drug.

3. The method of claim 1, wherein the combination of side effects of the drug includes depletion of at least one nutrient.

4. The method of claim 1, wherein administering the supplement comprises administering a supplement formulated not to interfere with a beneficial activity of the drug.

5. The method of claim 1, wherein administering the supplement comprises administering a supplement formulated not to interfere with an effect of the drug on a body of the individual.

6. The method of claim 1, wherein administering the supplement comprises administering the supplement in accordance with a dosing schedule that corresponds to an expected timing of side effects of the drug.

7. The method of claim 6, wherein administering the supplement comprises administering the supplement in accordance with a plurality of different times during the day.

8. The method of claim 7, wherein administering the supplement comprises:
administering the supplement at about a same time as administering the drug; and
administering the supplement at a time an effectiveness of the drug is expected to end.

9. The method of claim 8, wherein administering the supplement comprises:
administering a first supplement for addressing at least one first side effect of the drug at about the same time as administering the drug; and
administering a second supplement for addressing at least one second side effect of the drug at the time when the effectiveness of the drug is expected to end.

10. The method of claim 1, wherein administering the drug comprises administering prednisone, an anti-seizure drug, metformin, a beta-blocker, or an antipsychotic drug.

11. A drug-supplement combination, comprising:
a prescription drug capable of treating a medical condition; and
a supplement including a combination of ingredients capable of addressing a combination of side effects of the prescription drug.

12. The drug-supplement combination of claim 11, wherein the supplement includes ingredients available in the United States Pharmacopeia-National Formulary (USP-NF).

13. The drug-supplement combination of claim 11, wherein an amount of each ingredient in the supplement corresponds to an effective dose for addressing the at least one side effect of the prescription drug.

14. The drug-supplement combination of claim 11, wherein an amount of each ingredient in the supplement corresponds to a recommended daily allowance of that ingredient.

15. The drug-supplement combination of claim 14, wherein the supplement is formulated for a plurality of doses each day.

16. The drug-supplement combination of claim 11, wherein the prescription drug comprises prednisone, an anti-seizure drug, metformin, a beta-blocker, or an antipsychotic drug.

17. The drug-supplement combination of claim 11, wherein the supplement is formulated to address depletion of at least one nutrient by the prescription drug.

18. The drug-supplement combination of claim 11, wherein the supplement is formulated to address all treatable common side effects and all treatable very common side effects of the prescription drug.

19. The drug-supplement combination of claim 11, wherein the supplement is formulated not to interfere with a beneficial activity of the prescription drug.

20. The drug-supplement combination of claim 11, wherein the supplement is formulated not to interfere with an effect of the prescription drug on a body of an individual to whom the prescription drug is to be administered.
